(19) United States
(12) Patent Application Publication
(10) Pub. No.: US 2022/0217971 A1
(43) Pub. Date: Jul. 14, 2022

(54) GRAFT TRANSPORTATION AND IMPLANTATION SYSTEM AND METHOD FOR USE

(71) Applicant: EPIBONE, INC., Brooklyn, NY (US)

(72) Inventors: Sarindr BHUMIRATANA, Oceanside, NY (US); Eric Meade JEFFRIES, Warrington, PA (US); Brian MCKELLAR, Exton, PA (US)

(73) Assignee: EPIBONE, INC., Brooklyn, NY (US)

(21) Appl. No.: 17/609,843

(22) PCT Filed: May 8, 2020
(86) PCT No.: PCT/US20/32008

§ 371 Date: Nov. 9, 2021

Related U.S. Application Data
(60) Provisional application No. 62/846,277, filed on May 10, 2019.
(60) Provisional application No. 62/883,349, filed on Aug. 6, 2019.

(51) Int. Cl. A01N 1/02; A61F 2/00; A61F 2/30; A61F 2/46

(52) U.S. Cl. CPC A01N 1/0273; A61F 2/0095; A61F 2/30756; A61F 2/4644; A61F 2002/4662; A61F 2002/30766; A61F 2002/4658; A61F 2002/4659; A61F 2/4657

(57) ABSTRACT

A graft holder for securely transporting a graft, the graft holder including a base surface; and a cavity extended from the base surface and formed from one or more walls, the cavity configured so the graft is securely nested therewith when being transported between locations so as to minimize damage and/or impact to the graft during transport.

### CROSS-REFERENCE TO RELATED APPLICATIONS

This application claims priority to U.S. Provisional Application No. 62/883,349, filed on Aug. 6, 2019 and to U.S. Provisional Application No. 62/846,277, filed on May 10, 2019, the disclosure of which is herein incorporated by reference in its entirety.

### BACKGROUND

Cartilage injuries affect approximately one million Americans annually, resulting in more than 500K cartilage-related procedures. Current method or uses of treating cartilage injuries include debridement and microfracture, marrow stimulation, autologous chondrocyte implantation (ACI), matrix-induced autologous chondrocyte implantation (MACI), mosaicplasty, and osteochondral autografting and osteochondral allografting. There are at least 350,000 knee arthroplasties performed each year, with chondral lesions present in >60% of cases. The number of such procedures is forecasted to increase due to population growth, longevity and advances in diagnosis tools.

Cartilage lesions are frequently associated with other articular injuries, and can progress to joint degeneration and osteoarthritis (OA). OA affects about 20% of Americans, with 10% of those with OA having activity limitations. Annual costs from OA are estimated to exceed $130 billion. The World Health Organization ranked OA as the fourth condition in terms of impact on disease-free life-years and disability, while the Centers for Disease Control and Prevention determined that arthritis is the number-one cause of disability in the United States. Osteoarthritis even affects younger people as post-traumatic osteoarthritis (PTOA). PTOA is estimated to cause 12% of symptomatic arthritis of the hip and knee, with associated costs of over $3 billion per year. PTOA and other common cartilage injuries particularly affect the active duty military population, which has about ten times the incidence of all common cartilage injuries as compared to civilians. As an example, ACL and meniscus injuries affect 3.65 and ˜6.5 out of 1000 soldiers, respectively, every year. The corresponding rates for civilians are ˜0.34 and ˜0.45 out of 1000. There is a great need to treat PTOA and cartilage injuries in the military population and in younger people.

The most common procedures to treat osteochondral defects are microfracture, mosaicplasty, allograft or autograft osteochondral grafting, ACI, and, more recently, MACI. All techniques suffer from several drawbacks that are affected by size limitations, the patient's age, and quality of surrounding cartilage. Drawbacks include reduced biomechanical properties, long recovery, double surgeries and need for donor tissues. Success rates tend to be low and there is a high risk for failures 1-5 years post-surgery.

Autograft and allograft transplantations have been used to treat cartilage injuries. Autograft has been shown to be effective in lesions up to 3 cm in diameter, with good-to-excellent outcomes reported even among athletes. Allograft osteochondral transplantation has previously been utilized in combat soldiers, allowing them to return to their military position. However, allograft osteochondral transplantation has proven to be less successful in active duty military populations when compared to civilians. A retrospective review analyzed the effectiveness of allograft osteochondral transplantation in the knee in the active duty population, focusing on ability of patients to return to their status following the procedure. Although this method or use of surgery for large lesions of the knee has a good rate of success among civilian patients, it failed to ensure retention on active duty for injured soldiers, particularly when they occupy a physically demanding military position. Many patients treated by allograft osteochondral transplantation have not been able to remain on active duty in their previous role. There is a need for improved transplantation therapies for those in the military who would otherwise have few alternative options in the military. There is also a need for improved transplantation therapies for those who lead a comparably physically active lifestyle, such as professional and amateur athletes, firefighters and police officers.

Improvements are still needed in preparing cartilage for transplantation. For a long time, cartilage has been a major focus of the field of tissue engineering, both due to the strong clinical need, the avascular nature of this tissue and low cell density. Cartilage tissue engineering has progressed mostly by utilizing primary chondrocytes extracted from native cartilage. These highly active, yet phenotypically stable and mature, cells could produce cartilaginous matrix. Even simple culture systems enabled engineering of viable cartilaginous tissue constructs from primary chondrocytes that are a few millimeters thick. Also, cultivation in bioreactors with a dynamic flow environment yielded cartilage with similar biochemical composition and mechanical stiffness as parent cartilage. In cartilage tissue engineering, juvenile bovine chondrocytes have been used in conjunction with scaffolding materials to engineer cartilage with mechanical properties approximating those of native tissue. These method or uses however suffer from limited availability, and limited ability to generate functional cartilage from adult chondrocytes.

Finally, in the cartilage field generally, there is a need to avoid damage to the cartilage layers that can be used for certain procedures that have to be shipped between locations as well as corresponding pre- and post-shipping handling, and final insertion into a defect of a patient.

In view of the above, there is a need to resolve these and other problems with the art.

### SUMMARY

Described herein are improved graft implantation systems, method or uses, and one or more tools as shown and described herein, including each and every novel feature or combination of features disclosed herein.

In some examples, a graft holder is disclosed for securely transporting a graft. The graft holder can include a base surface; and a cavity extended from the base surface and formed from one or more walls, the cavity configured so the graft is securely nested therewith when being transported between locations so as to minimize damage and/or impact to the graft during transport.

In some examples, the holder is configured to provide protection to a top portion of the graft while allowing for fluid access to an entirety of the graft through one or more fluid passages and/or gaps in the cavity.

In some examples, the base surface is substantially planar.

In some examples, extended between the base surface and the cavity is a hole, wherein the hole is configured to remove the graft out of the cavity and through the hole.

In some examples, the cavity further comprises one or more gaps, wherein the graft can be securely and safely attached to the holder by being positioned through the gaps.

In some examples, the cavity is largely open to reveal the top of the graft to permit passage of a graft retrieval tool.

In some examples, the cavity further comprises one or more peripheral surfaces positioned in a corner or extremities of the one or more walls.

In some examples, the one or more peripheral surfaces are partially thicker, column-like reinforced structures that can both shield graft during transport as well as enhance structural integrity of holder.

In some examples, a diameter of the cavity is configured to house a plurality of different sized grafts during transport.

In some examples, the holder further comprises internal walls of the cavity taller than the graft when the graft is nested in the cavity.

In some examples, the cavity further comprises regions of vertical cavities configured to permit flow of transport fluid to access the graft when nested therewith.

In some examples, the holder is integrally formed from molded silicone.

In some examples, a method or use is disclosed. The method or use can include transporting graft contained in a graft holder; engaging a graft retrieval tool into the graft; measuring the graft to an approximate height; selecting one of a plurality of graft depth guides by matching the graft to one of the plurality of graft depth guides with a desired height associated with a patient defect; inserting a guide post through a dilator and drilling the guide post into a defect of the patient; sliding the selected depth guide down the guide post to check a defect depth; and removing the selected depth guide and the guide post.

In some examples, the removal tool is tweezers.

In some examples, the removal tool is a graft retrieval tool comprising an elongated member with a distal engaging surface to remove the graft from the graft holder.

In some examples, the method or use can include inserting or removing the graft from the defect while the graft is still attached to the graft retrieval tool; verifying the defect depth of the graft based on flushness with native cartilage of the defect; and if the defect depth is generally flush, then detaching the graft retrieval tool from the graft.

In some examples, a distal engaging surface of the graft retrieval tool is a threaded surface for connecting to the graft by threaded engagement engaging the graft, wherein the step of detaching the graft is done by unscrewing the threaded surface from the graft.

In some examples, the method or use can include inserting or removing the graft from the defect while the graft is still attached to the graft retrieval tool; verifying the defect depth of the graft based on flushness with native cartilage of the defect; and if the defect depth is generally proud, then removing the graft from the defect while the graft is still attached to the graft retrieval tool; further drilling to increase the defect depth; reinserting the graft into the defect for verifying the defect depth; and if the defect depth is generally flush, then detaching the graft retrieval tool from the graft.

In some examples, a distal engaging surface of the graft retrieval tool is a threaded surface for connecting to the graft by threaded engagement engaging the graft, wherein the step of detaching the graft is done by unscrewing the threaded surface from the graft.

In some examples, the method or use can include if the graft is countersunk into the defect, then estimating visually an amount the graft is countersunk into the defect; removing the graft from the defect while the graft is still attached to the graft retrieval tool, and then hydrating the graft and the graft retrieval tool; positioning the selected graft depth guide into the defect; and evaluating flushness of the selected depth guide with respect to native tissue of the defect.

In some examples, the step of hydrating comprises maintaining hydration of the graft is by submerging the graft and the graft retrieval tool in a container with saline solution.

In some examples, the method or use can include verifying flushness of the selected graft depth guide with native cartilage of the defect and if not flush, then selecting a bone shim for use with the graft and the selected graft depth guide; hydrating the selected bone shim; and positioning the selected bone shim into the defect.

In some examples, the step of hydrating the selected bone shim is by submerging the shim in a container with saline solution for at least about a minute.

In some examples, the step of positioning the selected bone shim into the defect comprises pushing, by a pair of forceps securely engaged with the selected bone shim, the selected bone shim into a bottom of the defect.

In some examples, the method or use can include evaluating whether the selected graft depth guide is countersunk with native cartilage of the defect and if countersunk, then removing the selected graft depth guide; selecting a second graft depth guide from the plurality of graft depth guides based on the defect depth; and evaluating a difference in the initially selected graft depth guide and the second graft depth guide.

In some examples, the method or use can include selecting a bone shim corresponding to the difference between the selected depth guide and the second depth guide.

In some examples, the defect is at an articular cartilage defect site.

In some examples, the defect depth is based on visual and physical evaluation in the defect.

In some examples, the method or use can include repeating drilling until satisfied with how the selected depth guide sits in the defect.

In some examples, the method or use can include hydrating the graft with the graft retrieval tool attached therewith in saline solution until positioning in the defect.

In some examples, the method or use can include removing the graft holder from a container using a tool having opposing surfaces for removably attaching to the graft holder and/or the graft.

In some examples, the method or use can include securely connecting the graft with one or more alignment features of the graft holder, the graft holder can include a contoured outer surface that forms a cavity with the one or more alignment features; and securely connecting one or more retention surfaces of the graft holder with a removal tool having opposed elongated members for affixing therewith, the one or more retention surfaces being disposed on the contoured outer surface.

In some examples, the method or use can include the step of measuring is performed by simultaneously holding both the graft and the selected depth guide.

In some examples, the method or use can include the step of measuring is performed by putting both graft and the selected depth guide on a generally flat surface; and visually comparing the selected depth guide with the graft.

In some examples, each depth guide comprises approximately 0.1 mm increments.

In some examples, a method or use is disclosed for evaluating depth of a graft for a defect. The method or use can include positioning one of a plurality of depth guides in the defect; and evaluating flushness of the selected depth guide with respect to native tissue of the defect.

In some examples, the depth guide can include a lower base section for measuring depth guide thickness; a handle section extended from an upper surface of the lower base section, the handle section having a diameter smaller than a diameter of the lower base section; and a lumen extended through the lower base and handle sections.

In some examples, the depth guide is asymmetric.

In some examples, the depth guide is an anatomical shape.

In some examples, the method or use can include measuring, with the depth guide, a depth of the defect.

In some examples, the step of measuring is performed by simultaneously holding both the graft and the selected depth guide.

In some examples, the step of measuring is performed by positioning the graft and the selected depth guide on a generally flat surface; and visually comparing the selected depth guide with the graft.

In some examples, if the defect and the selected depth guide are insufficiently flush, the method or use further comprises: drilling until sufficiently flush with how the selected depth guide sits in the defect.

In some examples, the defect is at an articular cartilage defect site.

In some examples, the selected depth guide comprises approximately 0.1 mm increments.

In some examples, a method or use is disclosed. The method or use can include transporting a replacement tissue contained in a tissue holder; inserting a guide post through a dilator and drilling the guide post into the defect site of the patient; engaging a retrieval tool into the replacement tissue; removing replacement tissue from a tissue holder; selecting one of a plurality of tissue depth guides by matching the replacement tissue to one of the plurality of tissue depth guides; drilling or cleaning the defect region by debriding; measuring the tissue defect depth by sliding the selected depth guide down the guide post; inserting the depth guide into the defect to check the tissue defect depth; and inserting the replacement tissue into the site of the tissue defect and disengaging the retrieval tool.

In some examples, a system for repairing a defect of a patient. The system can include one or more grafts; one or more graft holders for holding the one or more grafts; a container containing the one or more graft holders; a plurality of graft depth guides; a graft retrieval tool comprising an elongated member with a distal engaging surface for removably engaging the one or more grafts; and a plurality of bone shims to be used for depth correction of grafts in defect site.

In some examples, the one or more grafts can be a plurality of grafts. In some examples, the grafts are osteochondral grafts. The osteochondral grafts include tissue from allosource or xenosource, synthetic material, biological material, or engineered tissues. In some examples, each graft of the system is separately contained within a respective graft holder.

In some examples, the system includes a plurality of graft depth guides for measuring graft height of a selected graft of the plurality of osteochondral grafts, wherein each graft depth guide includes a lower base section for measuring depth guide thickness; a handle section extended from an upper surface of the lower base section, the handle section having a diameter smaller than a diameter of the lower base section; and a lumen extended through the lower base and handle sections.

In some examples, the handle section is substantially longer than the lower base section.

In some examples, the handle section is at least twice as long as the lower base section.

In some examples, the lumen is configured for being slid along a guide post.

In some examples, thicknesses of the lower base section of each graft depth guide ranges between approximately about 6.5 mm and 7.6 mm, whereby this range can depend on graft thickness.

In some examples, thicknesses of the lower base section of each graft depth guide ranges between approximately about 4 mm and 15 mm, whereby this range can depend on graft thickness.

In some examples, the plurality of graft depth guides comprises at least 10 separate graft depth guides, each graft depth guide can include a different graft depth thickness separated by approximately about 0.1 mm.

In some examples, each handle section comprises a unique reference symbol indicative of a corresponding graft depth guide thickness.

In some examples, the system also includes one or more bone shims which can include a centrally positioned aperture for slidably receiving a guide post.

In some examples, the distal engaging surface of the graft retrieval tool is a threaded surface for connecting to the graft by threaded engagement engaging the graft(s).

In some examples, the container is substantially cylindrical with an open upper end and a closed lower end, the container containing a saline or cell culture media solution for storing the graft.

In some examples, the holder is configured to transported with the graft securely connected therewith.

In some examples, the holder includes a contoured outer surface that forms a cavity having one or more alignment features configured to securely connect with the graft; and one or more retention surfaces disposed on the contoured outer surface for being securely connected with a removal tool having opposed elongated members for affixing therewith.

In some examples, the holder is integrally from silicone.

In some examples, the removal tool is tweezers.

In some examples, the removal tool is graft retrieval tool with a threaded surface for connecting to the graft by threaded engagement engaging the graft.

In some examples, the removal tool is a rod that can be inserted from the base surface through the hole to the cavity and push the graft out of the holder.

To the accomplishment of the foregoing and related ends, certain illustrative aspects are described herein in connection with the following description and the appended drawings. These aspects are indicative, however, of but a few of the various ways in which the principles of the claimed subject matter may be employed and the claimed subject matter is intended to include all such aspects and their equivalents. Other advantages and novel features may become apparent from the following detailed description when considered in conjunction with the drawings.

### BRIEF DESCRIPTION OF THE DRAWINGS

The above and further aspects of this invention are further discussed with reference to the following description in conjunction with the accompanying drawings, in which like numerals indicate like structural elements and features in various figures. The drawings are not necessarily to scale, emphasis instead being placed upon illustrating principles of the invention. The figures depict one or more implementations of the inventive devices, by way of example only, not by way of limitation.

FIG. 1 depicts example features used in a procedure described in this disclosure.

FIG. 2 depicts example features used in a procedure described in this disclosure.

FIG. 3A depicts an upper perspective view of an example depth guide used in the procedure of this disclosure.

FIG. 3B depicts a lower perspective view of an example depth guide used in the procedure of this disclosure.

FIG. 4A depicts a top view of an example depth guide used in the procedure of this disclosure.

FIG. 4B depicts a bottom view of an example depth guide used in the procedure of this disclosure.

FIG. 5A depicts an upper perspective view of an example graft holder used in the procedure of this disclosure.

FIG. 5B depicts a lower perspective view of an example graft holder used in the procedure of this disclosure.

FIG. 6A depicts a bottom view of an example graft holder used in the procedure of this disclosure

FIG. 6B depicts a top view of an example graft holder used in the procedure of this disclosure

FIG. 7A shows one example view of the procedure.

FIG. 7B shows one example view of the procedure.

FIG. 8A shows one example view of the procedure.

FIG. 8B shows one example view of the procedure.

FIG. 9 shows one example view of the procedure.

FIG. 10 shows one example view of the procedure.

FIG. 11 shows one example view of the procedure.

FIG. 12A shows one example view of the procedure.

FIG. 12B shows one example view of the procedure.

FIG. 13A is a table for one example embodiment for calculating change of depth guide.

FIG. 13B is a table for one example embodiment an estimated depth guide thickness.

FIG. 14 shows an example view of the procedure.

FIG. 15 is a schematic overview of one example method or use.

### DETAILED DESCRIPTION

Although example embodiments of the disclosed technology are explained in detail herein, it is to be understood that other embodiments are contemplated. Accordingly, it is not intended that the disclosed technology be limited in its scope to the details of construction and arrangement of components set forth in the following description or illustrated in the drawings. The disclosed technology is capable of other embodiments and of being practiced or carried out in various ways.

It must also be noted that, as used in the specification and the appended claims, the singular forms “a,” “an” and “the” include plural referents unless the context clearly dictates otherwise. By “comprising” or “containing” or “including” it is meant that at least the named compound, element, particle, or method or use step is present in the composition or article or method or use, but does not exclude the presence of other compounds, materials, particles, method or use steps, even if the other such compounds, material, particles, method or use steps have the same function as what is named.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method or use does not preclude the presence of additional method or use steps or intervening method or use steps between those steps expressly identified. Steps of a method or use may be performed in a different order than those described herein without departing from the scope of the disclosed technology. Similarly, it is also to be understood that the mention of one or more components in a device or system does not preclude the presence of additional components or intervening components between those components expressly identified.

As discussed herein, a “graft” may be a structural tissues such as bone, cartilage, osteochondral tissue, ligament, tendon, etc. Such grafts may be autologous (e.g., graft harvested from the patient's own body), allograft (e.g., cadaveric graft usually obtained from a tissue bank), xenograft (e.g., graft harvested from an animal donor), lab grown (by a tissue engineering company) or synthetic. In the case of synthetic, such bone components of the osteochondral graft can be made of hydroxyapatite or other naturally occurring and biocompatible substances with similar mechanical properties to bone.

As discussed herein, a “graft depth guide” includes a mechanical structure that is configured to serve as a reference to how the graft will fit into the defect site of a patient during the procedure. The graft depth guide can have predetermined dimensions, such as a predetermined depth configured to correspond to a particular defect depth.

As discussed herein, vasculature of a “subject” or “patient” may be vasculature of a human or any animal. It should be appreciated that an animal may be a variety of any applicable type, including, but not limited thereto, mammal, veterinarian animal, livestock animal or pet type animal, etc. As an example, the animal may be a laboratory animal specifically selected to have certain characteristics similar to a human (e.g., rat, dog, pig, horse, monkey, or the like). It should be appreciated that the subject may be any applicable human patient, for example.

As discussed herein, “operator” may include a doctor, surgeon, or any other individual or delivery instrumentation associated with delivery of the herein disclosed graft and corresponding tools to a surgical location on a patient (e.g., a knee).

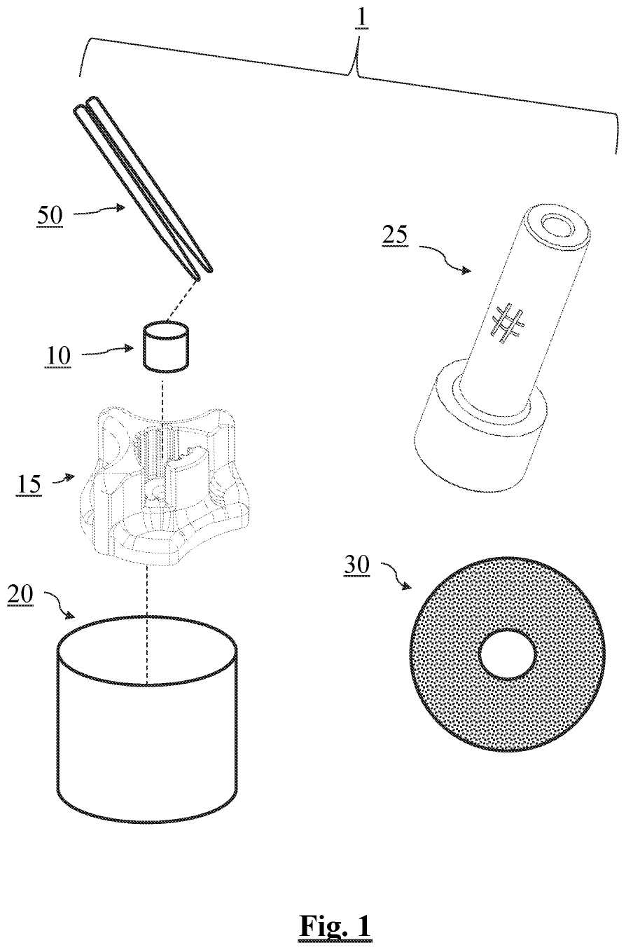

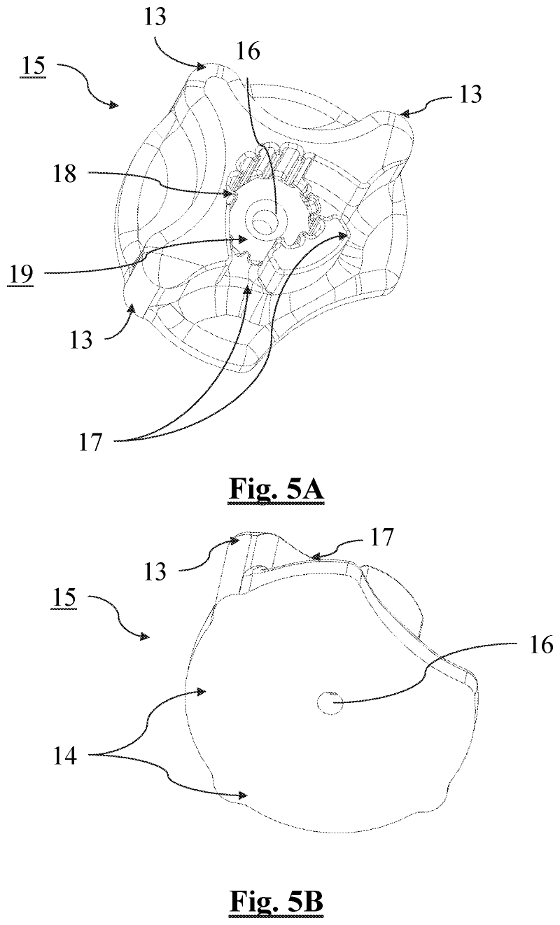

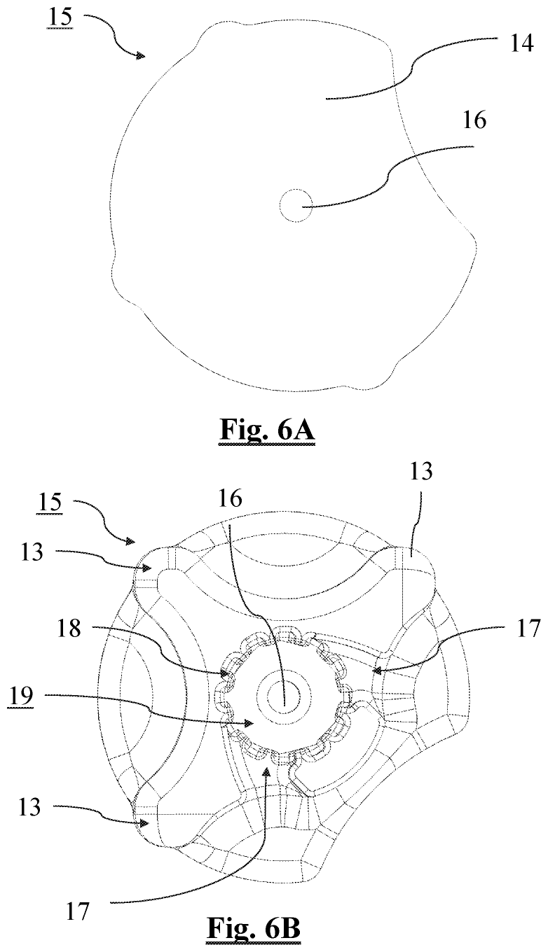

As an example, FIG. 1 depicts example features used in a system 1 for a procedure described in this disclosure. The depicted system 1 can include any number of osteochondral grafts 10 separately packed or together packed in a graft holder 15, in separate containers 20 or single container 20. It is understood that grafts 10 of this disclosure can be formed from cartilage and/or bone from another site in the knee (autograft), a tissue donor knee (allograft), or manufactured using tissue engineering principles. As can be seen, holder 15 can be transported with graft 10 stored therein, as shown more particularly in FIGS. 5A-6B. Holder 15 may be housed in container 20 for transporting between locations. Container 20 can house a liquid solution, such as a saline solution.

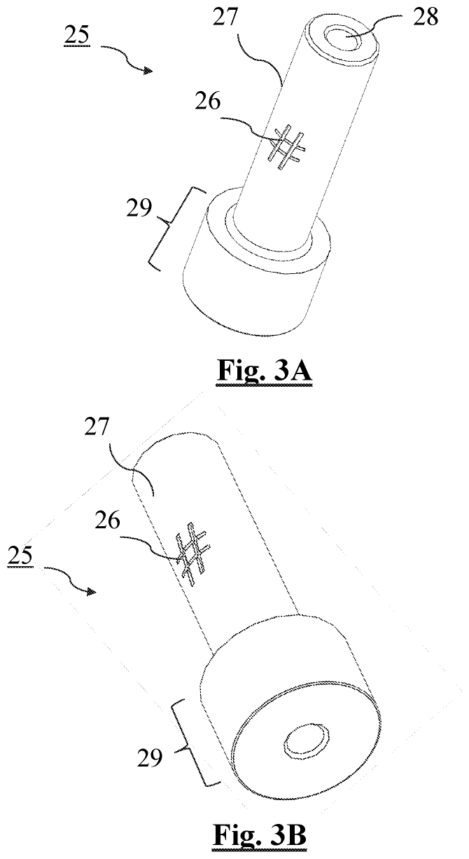

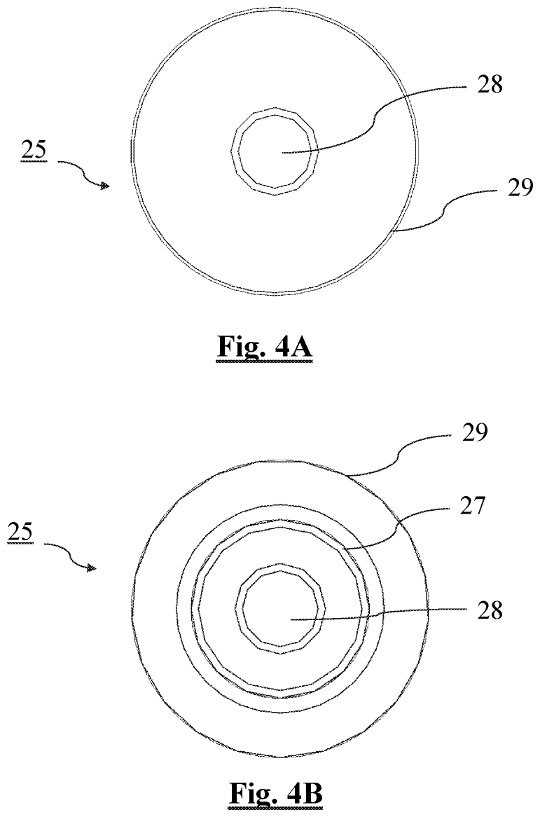

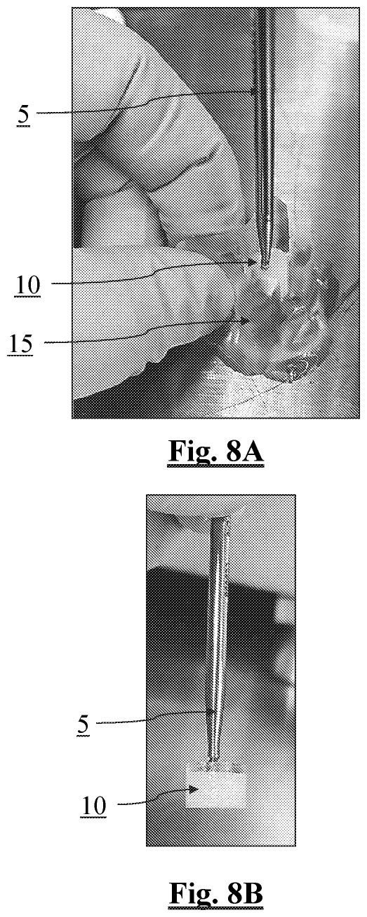

A graft retrieval tool 5 or removal tool 50 (e.g., tweezers) can be used in system 1 to affix or remove graft 10 from holder 15 as well as use graft 10 later in the procedure, as described more particularly in FIGS. 8A-10. As can be seen, tool 50 can include one or more elongated members with distal tips configured to attach to graft 10. In the case of tool 5, it too can include one or more elongated members with distal tips configured to attach to graft 10. System 1 can also include one or more depth guides 25 that can range in thickness based on the thickness of the osteochondral graft (e.g., 6.5-7.6 mm, 7.0-8.0 mm, 8.0-10.0 mm, etc.), as discussed more particularly in FIGS. 3A-4B.

The depicted system 1 can also include one or more bone shims 30 having one or more beveled and/or relatively planar features. The bone shim 30 depicted in FIG. 1 can be manufactured with different individual thickness(es) and can be used to correct depth of the grafts 10 during the procedure of this disclosure. The bone shim 30 shown in FIG. 1 is depicted with a centrally positioned aperture for a guide post to be translated therethrough. Bone shim 30 is displayed in FIG. 1 bevel side up.

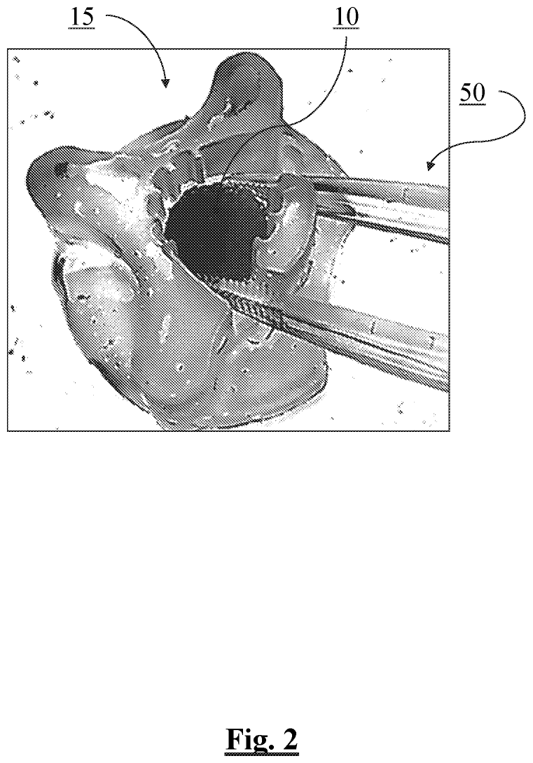

FIG. 2 depicts an example osteochondral graft 10 positioned in holder 15, which in the depicted example holder 15 can be formed of silicone. Graft 10 is shown with tool 50 affixed or otherwise connected therewith, whereby a removal tool 50 is shown with opposing elongated members holding the outer surface of graft 10 for removal from holder 15. Tool 50 can be used in system 1 to affix or remove graft 10 from holder 15. Tool 50 can be one or a pair of opposing elongated members with tapered and/or sharpened angled distal tips for affixing to graft 10 and/or holder 15. In one example, tool 50 can be a set of for picking up graft 10. However, tool 50 could also be one more tongs, pincers, forceps, or scissors-like pliers used to grab or hold graft 10.

FIG. 3A depicts an upper perspective view of depth guide 25 used in the procedure of this disclosure. FIG. 3B similarly depicts a lower perspective view of depth guide 25. Depth guide 25 can be have a wider base section 29 that has a predetermined depth guide thickness. An elongated handle section 27 can extend therefrom, whereby section 27 can include a small diameter than section 29. A lumen 28 may extend through sections 27 and 29 for sliding along guide post 35, as discussed herein. FIG. 4A depicts a top view of depth guide 25 while FIG. 4B depicts a bottom view of depth guide 25 clearly showing lumen 28 extended between sections 27 and 29.

Section 27 may include one or more reference symbols 26 indicative of the depth guide thickness associated with section 29. For example, system 1 can include a plurality of guides 25 to collectively have a thickness of respective sections 29 between 6.5-7.6 mm. Optionally, the depth guide thickness of section 29 can be adjustable as needed or required. For example and without limitation, section 29 could include one or more layers capable of being added or removed thereon to adjust the depth guide thickness.

FIG. 5A depicts an upper perspective view of graft holder 15 used in the procedure of this disclosure while FIG. 5B depicts a lower perspective view of graft holder 15. Similarly, FIG. 6A depicts a bottom view of graft holder 15 while FIG. 6B depicts a top view of graft holder 15. Holder 15 is configured to minimize damage to the graft 10 during transport. During use, the graft 10 can sit in the holder 15, with the holder walls taller than the graft 10 when nested therewith, whereby the walls are configured to protect of the graft 10 from impact during transport. The diameter of the graft holder 15 can be designed to house various diameter graft sizes during transport. Holder 15 can be molded silicone and can include a base surface 14 that can be substantially planar or flat. Holder 15 can also include surfaces 13 positioned in the corner or extremities thereof, including being formed as partially thicker, column-like reinforced structures that can both shield graft 10 during use as well as enhance structural integrity of holder 15. Cavity 19 can hold graft 10 and the diameter of cavity 19 can be designed based on the diameter of graft 10.

Cavity 19 can also include some regions of vertical cavities 18 that can allow for transport fluid to access the graft 10. In the bottom of cavity 19, is a hole 16 or aperture that is through the bottom of the holder. This hole 16 can serve as a secondary way to remove the graft by the graft up an out of cavity 19, through hole 16. Cavity 19 can include a gap 17. In some examples, graft 10 can be securely and safely attached to holder 15 by placing graft 10 in cavity 19 using tweezers 50 or equivalent tool with gaps 17.

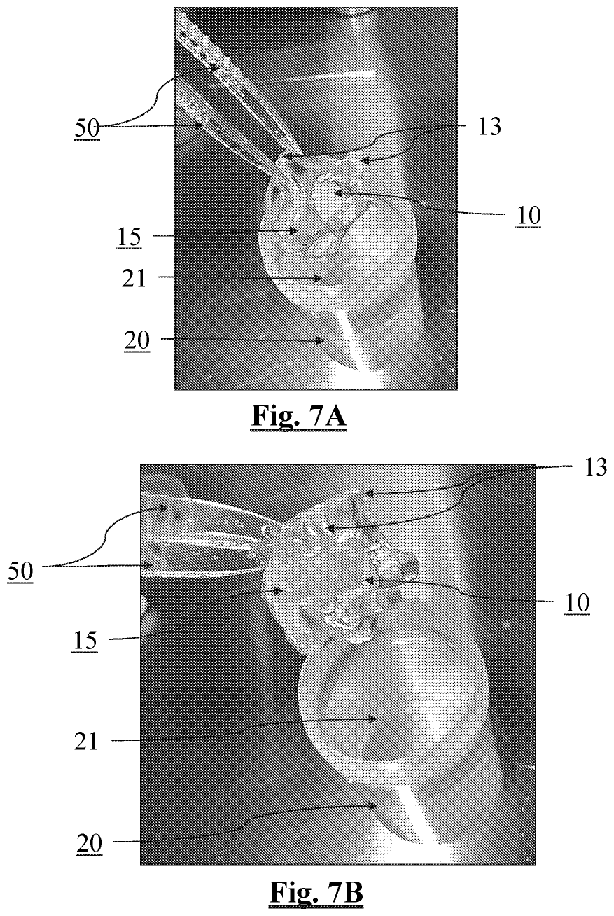

In FIGS. 7A-B, holder 15 is shown being removed from container 20, for example, using tool 50. Container 20 can be seen as being cylindrical with a liquid solution 21 (e.g., saline solution or cell culture media) disposed therein during housing of holder 15 and graft 10. In FIGS. 7A-B, tool 50 can be seen attached to surface 13 as holder 15 is being removed from container 20 while graft 10 is visibly stored in a recess. As previously discussed in FIGS. 6A-6B graft 10 can be securely and safely attached to holder 15 in said recess by placing graft 10 in cavity 19 by using tweezers 50 or equivalent tool and utilizing the gaps 17 in cavity 19.

In FIGS. 8A-B, the graft retrieval tool 5 is shown being screwed into the graft 10 while the operator is holding the holder 15. In this example, a single tool 5 is provided with a substantially elongated section that terminates in a distal engaging surface to remove the graft from the graft holder (e.g., a threaded distal end). The threaded distal end of tool 5 can be capable of being connecting to the graft by threaded engagement engaged with graft 10, which in FIG. 8B is shown as a relatively squared, three-dimensional structure. Operators are careful not to screw down through the bottom of the graft 10 during the depicted steps and thereafter, the graft 10 is measured (e.g., using a ruler) to approximate its height.

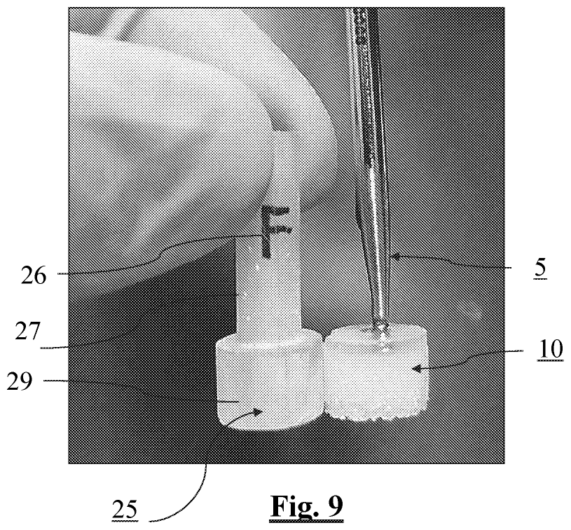

In FIG. 9, the graft 10 is shown being matched to the depth guide 25 with the desired height. The depth guide 25 shown in FIG. 9 is representative of how the graft 10 will sit in the defect. For example, matching as shown can be done while holding both graft 10 and guide 25 or alternatively both putting graft 10 and guide 25 both on a generally flat surface to compare.

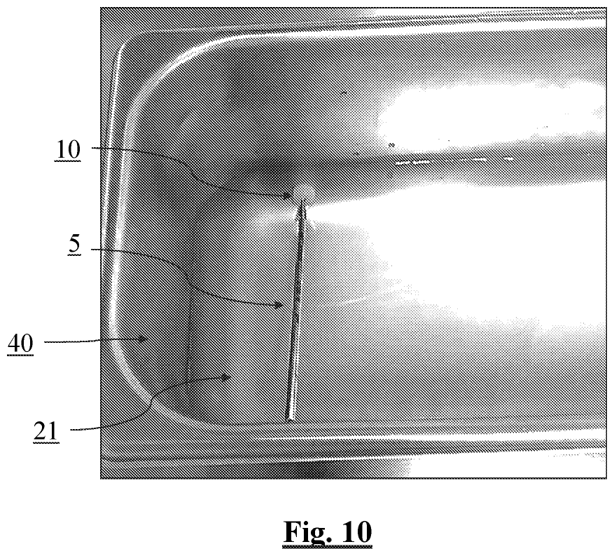

In FIG. 10, the graft 10 is submerged with the graft retrieval tool 5 in an example surgical pan 40. In the depicted example, pan 40 can include solution 21 and tool 5 and graft 10 can remain in pan 40 as shown until both are needed by the operator. Thereafter, the guide post 35 can be inserted through a dilator and the guide post 35 drilled into the patient (e.g., at the articular cartilage defect site). The defect is then created and drilled, whereby it is preferable to under drill depth rather than over drill.

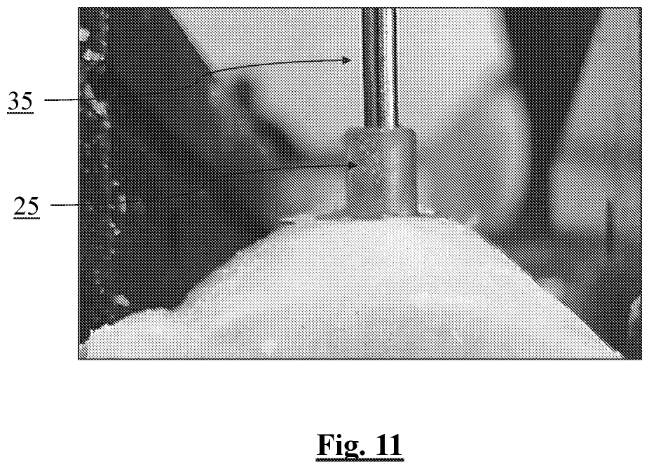

In FIG. 11, the selected depth guide 25 is slid down the guide post 35 to check defect depth based on visual and physical evaluation in the defect itself. Drilling is repeated until the operator is satisfied with how the depth guide 25 sits in the defect. Depth guide level in the defect can be assessed by visual and/or in combination with physical evaluation of depth guide level relative to adjacent native tissue.

In some examples, if the defect is over drilled and depth guide is countersunk, then the depth guide 25 is removed and a new depth guide having different dimensions is selected to measure the actual depth of the defect. Further, the defect is evaluated as to how much deeper it is than the original depth guide selected. For example, depth guide 25 labeled as “C” may have been originally selected based on the graft height, but the defect now fits a depth guide 25 labeled as “F”. This would suggest a change of 3 depth guides. If the defect is not over drilled and the depth guide is not countersunk, then the depth guide 25 and the guide post 35 can be removed. The graft 10 and tool 5 can then be retrieved from pan 40 and positioned in the defect.

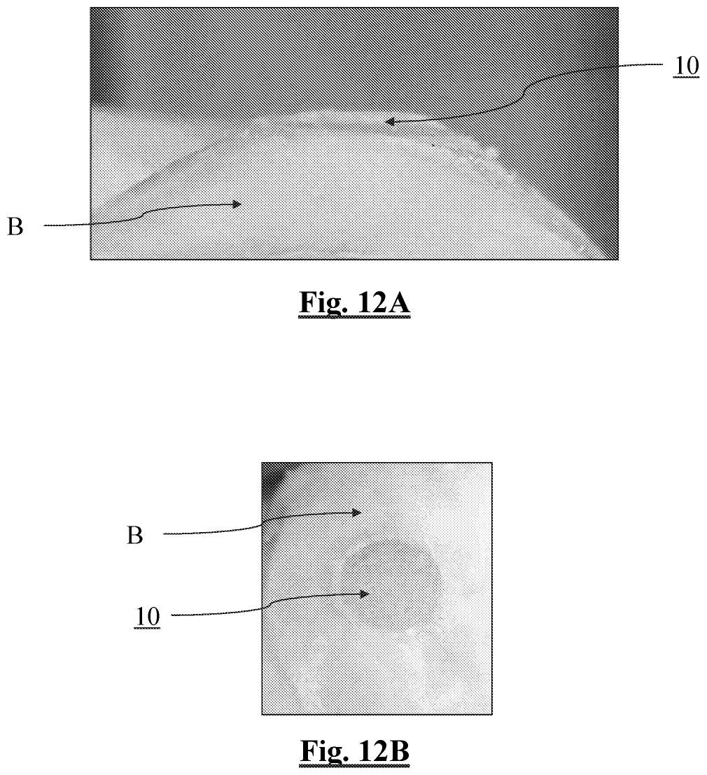

In FIGS. 12A-B, the depth of the graft 10 is shown being checked based on flushness with native cartilage of the patient. If the operator is satisfied with the graft 10 depth, the threaded distal end of tool 5 is unscrewed, and implantation is considered complete. However, if the operator is not satisfied, and it is deemed that the graft 10 is proud, the graft 10 is removed from the defect while still being held by tool 5. With or without reinserting the guide post 35, the operator can conservatively drill to increase the depth of the defect. Then the operator can check the depth by reinserting the graft 10 into the defect. Drilling and checking can be repeated with the graft 10 until satisfied with the graft height relative to the native cartilage.

If the graft is countersunk into the defect, then the amount the graft is countersunk is visually estimated. The graft can be removed from the defect while still being held by the graft retrieval tool and then returned to the pan 40. The originally selected depth guide can be placed back into the defect. The depth guide flushness can then be evaluated by the operator with the native tissue. If the depth guide is flush in the defect, then visual estimation is used by the operator to select a bone shim, as shown in FIG. 1. The selected bone shim is placed in the saline solution for approximately about 1 minute, though other times are contemplated that could be less or more than a minute. A separate saline container can be used from the graft holding pan. The bone shim can then be placed, bevel down, into the defect. This can be done using forceps to push the shim to the bottom of the defect. The graft can then be placed back into the defect and the flushness can be assessed with the native tissue. If satisfied with the graft depth, the operator can unscrew the graft retrieval tool.

As previously discussed, if the defect is over drilled and depth guide is countersunk, then the depth guide is removed, and anew depth guide is selected based on depth of the defect. Further, the defect is evaluated as to how much deeper it is than the original depth guide selected. For example, if depth guide A was originally selected based on the graft height, but the defect now fits guide D. This is a change of 3 depth guides.

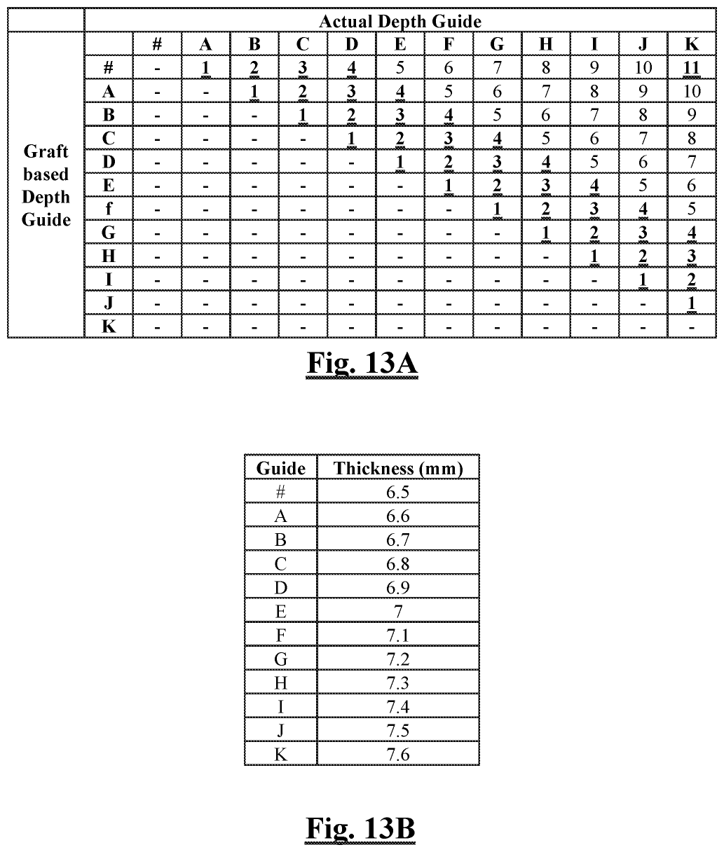

FIG. 13A is a table for one example embodiment for calculating change of depth guide 25. Similarly, FIG. 13B is a table for one example embodiment of an estimated depth guide thickness associated with sections 29 of guides 25. Each of these tables can be used for evaluating depth and/or selecting depth guides and corresponding elements in connection with the herein described solution.

With this in mind, a bone shim 30 can be selected corresponding to the difference between the original and newly selected depth guide 25. In some examples, bone shims 30 are labeled corresponding to their relative difference in depth.

Once selected, the selected bone shim 30 can be hydrated and placed in solution 21 for approximately about 1 minute. However, bone shim 30 can be placed in the solution 21 for longer or shorter than 1 minute as needed or required. The bone shim 30 can then be placed, oriented bevel down, into the defect. The bone shim 30 with its aperture in the middle can be placed over the guide post 35 and the depth guide 25 can be inserted to determine the flushness of the top surface. Tool 50 can be used to push the selected shim 30 to the bottom of the defect. The graft 10 can then be placed back into the defect (after removal of the guide post 35) and the flushness can be assessed with the native tissue. If satisfied with graft depth, the operator can then unscrew the threaded distal end of the graft retrieval tool 5 and implantation is considered complete.

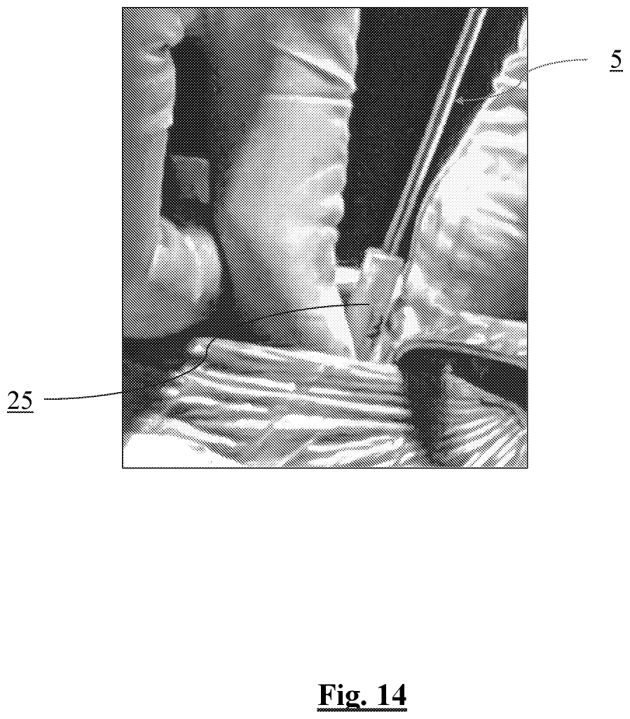

In FIG. 14, another view is shown of the selected depth guide 25 being slid over guide post into the defect. As can be seen, the operator is physically feeling depth guide with his fingers to evaluate flushness of depth guide 25 with native tissue.

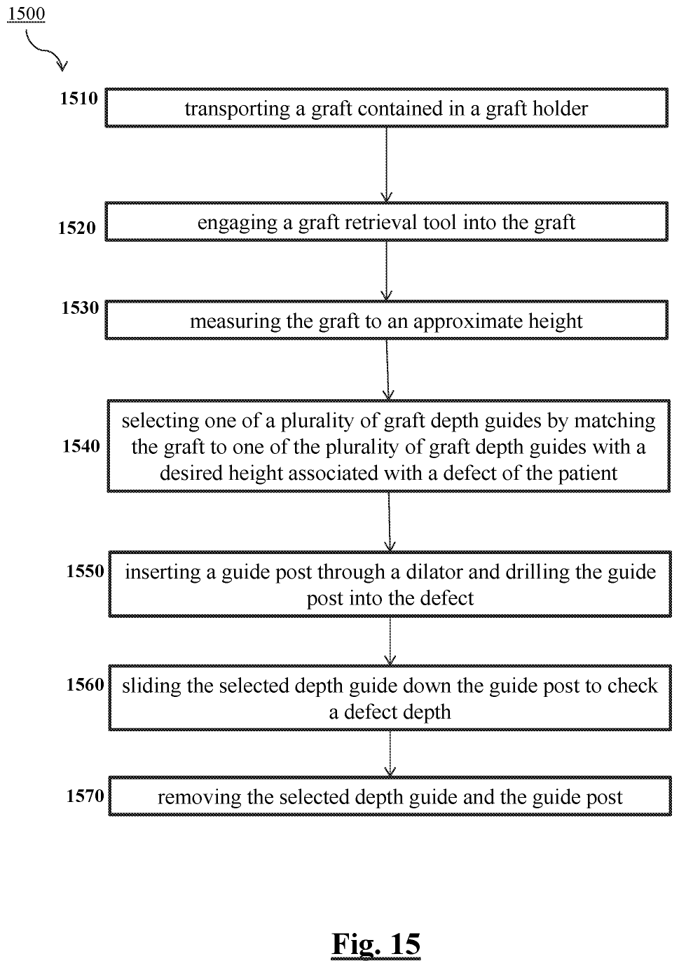

Turning to FIG. 15, an example method or use 1500 using grafts of this disclosure is shown. The method or use 1500 can include 1510 transporting a graft contained in a graft holder; 1520 engaging a graft retrieval tool into the graft; 1530 measuring the graft to an approximate height; 1540 selecting one of a plurality of graft depth guides by matching the graft to one of the plurality of graft depth guides with a desired height associated with a defect of the patient; 1550 inserting a guide post through a dilator and drilling the guide post into the defect; 1560 sliding the selected depth guide down the guide post to check a defect depth; and 1570 removing the selected depth guide and the guide post. In some examples, the method or use 1500 can include inserting the graft retrieval tool and graft into the defect and disengaging the graft retrieval tool from the graft.

The specific configurations, choice of materials and the size and shape of various elements can be varied according to particular design specifications or constraints requiring a system or method or use constructed according to the principles of the disclosed technology. Such changes are intended to be embraced within the scope of the disclosed technology. The presently disclosed embodiments, therefore, are considered in all respects to be illustrative and not restrictive. It will therefore be apparent from the foregoing that while particular forms of the disclosure have been illustrated and described, various modifications can be made without departing from the spirit and scope of the disclosure and all changes that come within the meaning and range of equivalents thereof are intended to be embraced therein.

### EXAMPLES

Various aspects of the disclosed solution may be still more fully understood from the following description of some example implementations and corresponding results. Some experimental data is presented herein for purposes of illustration and should not be construed as limiting the scope of the disclosed technology in any way or excluding any alternative or additional embodiments.

One example of certain implementations of the disclosed technology and corresponding results will now be described, in which an example graft specimen was placed in an example graft holder, and a fluid filled shipping container on a shaker at 200 RPM for approximately an hour, followed by 100 RPM for approximately 24 hours, and then returned to 200 RPM for approximately an hour. Each example graft specimen remained positioned safely in their respective graft holders following completion of the shaking and respective shaking time. Further, the operator in each experiment was successful with the example distal engaging surface of the example removal tool to the respective example graft specimen during each experiment while the specimen remained in the graft holder.

1-66. (canceled)

67. A graft holder for securely transporting a graft, the graft holder comprising:
a base surface; and
a cavity extended from the base surface and formed from one or more walls, the cavity configured so the graft is securely nested therewith when being transported between locations so as to minimize damage and/or impact to the graft during transport.

68. The graft holder of claim 67, wherein the holder is configured to provide protection to a top portion of the graft while allowing for fluid access to an entirety of the graft through one or more fluid passages and/or gaps in the cavity.

69. The graft holder of claim 67, wherein extended between the base surface and the cavity is a hole, wherein the hole is configured to remove the graft out of the cavity and through the hole.

70. The graft holder of claim 67, wherein the cavity further comprises one or more gaps, wherein the graft can be securely and safely attached to the holder by being positioned through the gaps.

71. The graft holder of claim 67, wherein the cavity is largely open to reveal a top portion of the graft.

72. The graft holder of claim 67, wherein the cavity further comprises one or more gaps configured to permit passage of a graft retrieval tool, and one or more peripheral surfaces positioned in a corner or extremities of the one or more walls.

73. The graft holder of claim 72, wherein the one or more peripheral surfaces are partially thicker, column-like reinforced structures configured to shield the graft during transport and enhance structural integrity of holder.

74. The graft holder of claim 67, wherein a diameter of the cavity is configured to house a plurality of different sized grafts during transport.

75. The graft holder of claim 67, wherein the holder further comprises internal walls of the cavity taller than the graft when the graft is nested in the cavity.

76. The graft holder of claim 67, wherein the cavity further comprises regions of vertical cavities configured to permit flow of transport fluid to access the graft when nested therewith.

77. A system for repairing a defect of a patient, the system comprising:
one or more grafts;
one or more graft holders for holding the graft;
a container containing the one or more graft holders;
a plurality of graft depth guides;
a graft retrieval tool comprising an elongated member with a distal engaging surface for removably engaging the one or more grafts; and
a plurality of bone shims to be used for depth correction of graft in defect site.

78. The system of claim 77, wherein each of the plurality of graft depth guides comprises:
a lower base section for measuring depth guide thickness;
a handle section extended from an upper surface of the lower base section, the handle section having a diameter smaller than a diameter of the lower base section; and
a lumen extended through the lower base and handle sections.

79. The system of claim 78, wherein a thicknesses of the lower base section of each graft depth guide ranges between about 4 mm and 15 mm.

80. The system of claim 78, wherein the plurality of graft depth guides comprises at least 10 separate graft depth guides, each graft depth guide comprising a different graft depth thickness separated by about 0.1 mm.

81. The system of claim 80, wherein each handle section comprises a unique reference symbol indicative of a corresponding graft depth guide thickness.

82. The system of claim 77, further comprising: one or more bone shims comprising a centrally positioned aperture for slidably receiving a guide post.

83. The system of claim 77, wherein the distal engaging surface of the graft retrieval tool is a threaded surface for connecting to the graft by threaded engagement engaging the graft.

84. The system of claim 77, wherein the container is substantially cylindrical with an open upper end and a closed lower end, the container containing a saline or cell culture media solution for storing the graft.

85. The system of claim 77, wherein the holder comprises:
a contoured outer surface that forms a cavity having one or more alignment features configured to securely connect with the graft; and
one or more retention surfaces disposed on the contoured outer surface for being securely connected with a removal tool having opposed elongated members for affixing therewith.

86. A method for use, the method comprising:
transporting a replacement tissue contained in a tissue holder;
inserting a guide post through a dilator and drilling the guide post into a site of a tissue defect of a patient;
engaging a retrieval tool into the replacement tissue;
removing replacement tissue from a tissue holder;
selecting one of a plurality of tissue depth guides by matching the replacement tissue to one of the plurality of tissue depth guides;
drilling or cleaning the defect region by debriding;
measuring the tissue defect depth by sliding the selected depth guide down the guide post;
inserting the depth guide into the defect to check the tissue defect depth; and
inserting the replacement tissue into the site of the tissue defect and disengaging the retrieval tool.
